(19) United States
(12) Patent Application Publication
(10) Pub. No.: US 2020/0030592 A1
(43) Pub. Date: Jan. 30, 2020

(54) Apparatus For Administering Liquids To A Human Intravenously Via An Implanted Port

(71) Applicant: Stephen T. Cheche, North Andover, MA (US)

(72) Inventor: Stephen T. Cheche, North Andover, MA (US)

(21) Appl. No.: 16/078,556

(22) PCT Filed: Feb. 22, 2017
(86) PCT No.: PCT/US2017/018838

§ 371 Date: Aug. 21, 2018

Related U.S. Application Data
(60) Provisional application No. 62/298,255, filed on Feb. 22, 2016.

(51) Int. Cl. A61M 39/02; A61M 5/142; A61M 39/10

(52) U.S. Cl. CPC A61M 39/0208; A61M 5/14248; A61M 5/158; A61M 39/1011; A61M 5/14276

(57) ABSTRACT

Described is an apparatus for administering liquids to a human intravenously via an implanted port. The apparatus includes a primary catheter in fluid communication with a hollow needle and connectable to a bag for providing liquid; a push-button mechanism coupled to the hollow needle and configured so that depressing the push button mechanism inserts the hollow needle into the implanted port to provide liquid communication from the primary catheter, via the hollow needle, to the implanted port; and the primary catheter embedded within a flexible, polymer sheath, wherein the flexible polymer sheath has a removable cover on a front surface to protect the push button mechanism and adhesive on at least a portion of the back surface, the adhesive being covered with a removable backing.

### RELATED APPLICATION

This application claims the benefit of U.S. Provisional Application No. 62/298,255 filed on Feb. 22, 2016. The entire teachings of the above application are incorporated herein by reference.

### BACKGROUND

Often times, rapid infusion of liquid into the human body is necessary or desirable. Persons with cancer or other medical conditions often require frequent intravenous access for the administration of medication and fluids. Wounded soldiers on the battlefield may need fluids, medications and even blood intravenously before they reach definitive treatment in a hospital. Athletes often sweat and need fluid hydration and electrolyte replacement in order to maintain a high level of athletic performance. Any and all of these individuals would benefit from an implanted venous catheter port.

### SUMMARY OF THE INVENTION

Described herein is an apparatus for administering liquids to a human intravenously via an implanted port. The apparatus is affixed to the skin directly over the implanted intravenous port. The apparatus includes a primary catheter in fluid communication with a hollow needle and connectable to a bag for providing liquid. A push-button mechanism is coupled to the hollow needle and configured so that depressing the push button mechanism inserts the hollow needle through the skin into the implanted port to provide liquid communication from the primary catheter, via the hollow needle, to the implanted port. The primary catheter is embedded within a flexible, polymer sheath. The flexible polymer sheath has a removable cover on a front surface to protect the push button mechanism. The flexible polymer sheath also has adhesive on at least a portion of the back surface. The adhesive is covered with a removable backing. The apparatus can be adhered to the skin and applied over the implanted port, and can remain adhered to the skin for an extended duration of time.

In some instances, the apparatus also includes multiple secondary catheters in fluid communication with the primary catheter via a manifold. In some instances where the inside walls of the catheter are expected to come into contact with blood, the inner surfaces of one or more of the primary and secondary cathethers can be impregnated or coated with heparin in order to prevent or reduce coagulation or clotting of blood. In some instances, there can be two, three, or more secondary catheters. The multiple catheter lumens can be formed of a wide variety of materials, including silicone or polyurethane. Each lumen of the multiple catheter lumens can have a port connector, which can be a vacuum port connector.

The push button mechanism can couple the hollow needle to a push button by a spring. Typically, the spring is coupled to a locking component and configured to lock the needle into the implanted port when the push button is depressed. The push button mechanism can further include an antibiotic disc seated at the base of the push button mechanism and within the push button mechanism. Typically, the adhesive does not cover the antibiotic disc.

The hollow needle can have a small hole on an upper surface that is connected to a reservoir near the top of the push button via a channel so that blood can flow from the hollow needle to the reservoir, thereby indicating that the hollow needle has punctured the implanted port.

In some instances, the removable backing can have at least first and second sections that can be separately removed. The first section can cover a portion of the adhesive configured to adhere the antibiotic disc to the skin directly over the implanted port. The second section can cover a portion of the adhesive configured to adhere to skin surrounding the implanted port.

Also described herein is an apparatus for providing liquid intravenously to a human. The apparatus can include a liquid bag having a first, front flap that is configured to be pulled open to provide access to intravenous tubing that is connected from the liquid bag into a flush-flow box and a second, back flap that is configured to be opened to provide access to a rear compartment. The flush-flow box can have an entry pathway in liquid communication with a first switch, and a second pathway in liquid communication with a second switch, as well as a common outlet tubing in liquid communication with both switches.

The intravenous tubing can be connected from the bag to the flush-flow box connected to the first switch that operates to flush the intravenous tubing and provide liquid to flow to a port. The second switch in the flush-flow box has a vacuum component, connects to the common outlet tubing, and operates to aspirate the port prior to infusion to verify proper venous access. The rear compartment can include one or more of gloves and an antiseptic wipe.

Also described herein is a push-button mechanism. The push-button mechanism can include a housing, a screw-top cap, and a sheath that provides an inlet for a catheter or needle. The housing can enclose a button, a needle that couples a side to the lower opening, an antibiotic disc seated at a lower opening, a locking mechanism positioned above the antibiotic disc, and a spring positioned between the button and the housing component. The screw-top cap can cover the button. The housing can have many shapes, including cylindrical.

Also described herein is a flush-flow box. The flush-flow box can include an entry pathway in liquid communication with a first switch, and a second pathway in liquid communication with a second switch, and a common outlet tubing in liquid communication with both switches. The flush-flow box can further include an entry for intravenous tubing in fluid communication with the first switch, wherein the first switch operates to flush the intravenous tubing and provide liquid to flow to a port. The flush-flow box can also include a second switch having a vacuum component that connects to common outlet tubing and operates to aspirate a port prior to infusion to verify proper venous access.

Rapidly infusing liquids into the human body can replace bodily fluids lost due to injury, sweating, vomiting, or other causes. The devices described herein provide a number of advantages. The port is implanted by a one-time, minimally invasive surgical procedure, and can subsequently remain under the skin for years with little to no maintenance. Once inserted, the port has a low profile on the body surface. Then, the apparatus for administering liquids can be easily adhered to the implanted port by the person with the implanted port at any particular time and place, as necessary. Thus, the device is highly portable. When necessary, the device can be activated to provide rapid infusion of liquids, which can in some instances contain medicines, which can be particularly advantageous for a soldier in theater. Thus, the apparatus can be worn without being engaged and is ready for activation when needed. In embodiments having a plurality of catheters, the apparatus can be used to administer liquids, blood, and life-saving medication through the cathethers. As a result, more efficient patient care, lower infection rates, fewer peripheral IV line problems (e.g., infiltration of IV fluid into surrounding tissue, infection of peripheral lines, dislodging of peripheral lines), and rapid hydration can be achieved.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a schematic illustration of a front view of an apparatus for administering liquids to a human intravenously via an implanted port.

FIG. 1B is a schematic illustration of a back view of an apparatus for administering liquids to a human intravenously via an implanted port.

FIG. 1C is a schematic illustration of a cross-sectional view of an apparatus for administering liquids to a human intravenously via an implanted port.

FIG. 2A is a schematic illustration of an outside view of a push-button access needle mechanism and associated cover.

FIG. 2B is a schematic illustration of the interior of the push-button access needle mechanism and associated cover.

FIG. 3A is a schematic illustration of successive, front views of an intravenous fluid bag having a peel-down compartment.

FIG. 3B is a schematic illustration of successive, back views of an intravenous fluid bag having a peel-down compartment.

FIG. 4 is a schematic illustration of a flush-flow box.

### DETAILED DESCRIPTION OF THE INVENTION

A description of example embodiments of the invention follows. Generally, the invention is directed to an apparatus for administering liquids to a human intravenously via an implanted port. Prior to using the apparatus, an embedded intravenous port is implanted into a human. A variety of suitable implantable ports are commercially available. One such implantable port is PORT-A-CATH (Smiths Medical, Dublin, Ohio, USA). Another suitable implantable port is the VORTEX line of implantable ports (Angiodynamics, Latham, N.Y., USA). These ports can be surgically implanted under the skin and into a central venous blood vessel of a human. The most common central venous ports access the subclavian veins and the femoral veins. The great saphenous veins can also be accessed. Generally speaking, the port then remains implanted until it is desirable to use or remove the implanted port. The implanted port can be used with an apparatus for administering liquids, as described herein.

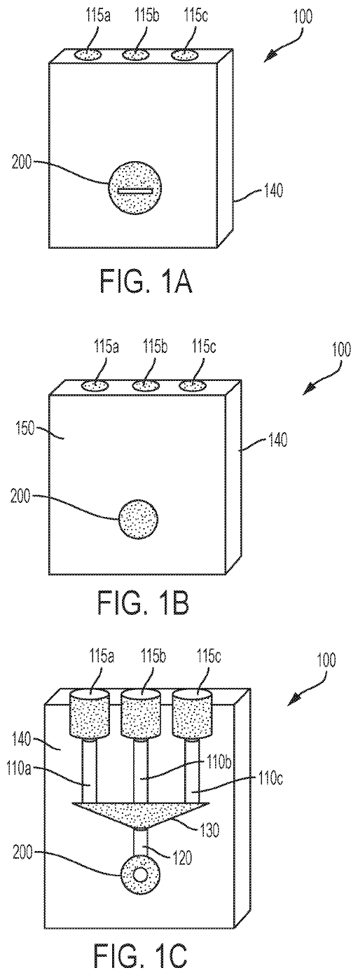

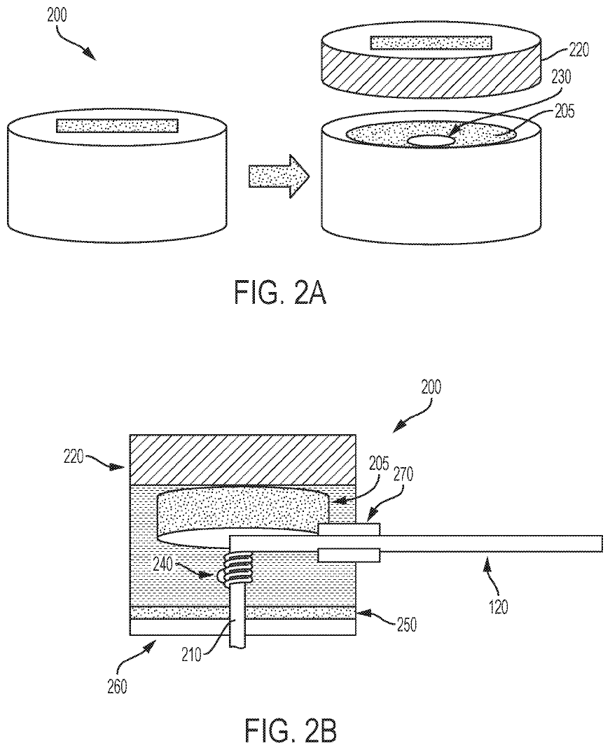

FIGS. 1A-C illustrate an apparatus 100 for administering liquids to a human intravenously via an implanted port. As illustrated, the apparatus 100 has multiple secondary catheters 110a-c that are in fluid communication with a primary catheter 120 via a manifold 130. The primary catheter 120 is also in fluid communication with a hollow needle 210 (FIG. 2B). A push-button mechanism 200 (see FIGS. 1A-C, 2A, and 2B) is coupled to the hollow needle 210. Depressing the push button 205 causes a corresponding force to be applied to the hollow needle 210, thereby inserting the hollow needle 210 into an implanted port (not shown) situated directly below the hollow needle 210. As a result, liquid communication from the primary catheter 120, via the hollow needle 210, to the implanted port is established.

The multiple secondary catheters 110a-c, manifold 130, and primary catheter 120 can be embedded within a flexible sheath 140, which is typically made of a medical grade polymer, such as polyethylene. Typically, the push-button mechanism 200 is also embedded within the flexible, polymer sheath 140. A removable cover 220 disposed above the push button 205 protects the push button 205 from being accidentally depressed. The removable cover 220 is typically a screw-top cover. The hollow needle 210 is coupled to the push button 205 via a spring 240, which, in turn, is coupled to a locking component 250 and configured to lock the needle 210 into the implanted port once the push button 205 has been depressed. In general, push button 205, spring 240, and locking component 250 function similarly to a ball-point pen, whereby depressing the push button 205 causes insertion of the hollow needle 210 into the implanted port. In the embodiment illustrated in the figures, the hollow needle 210 is L-shaped and joined to the primary catheter 120 at sheath 270, though other configurations are possible. The sheath 270 can be made of a variety of suitable materials, such as silicone or medical-grade materials.

In some embodiments, a reservoir 230 is in fluid communication with the hollow needle 210. Upon depressing the push button 205, blood can flow upward through the hollow needle 210 and into the reservoir 230 via capillary action. The presence of blood in the reservoir 230 indicates that the hollow needle has punctured the implanted port.

The multiple secondary catheters 110a-c are preferably formed of a medically-safe material, such as silicone or polyurethane, though other materials are acceptable as well. In some instances, the multiple catheter lumens 110a-c are impregnated with a heparin solution, which reduces or prevents coagulation or clotting of blood. Typically, the multiple lumen catheter has two or three lumens, though other arrangements are possible. Each lumen 110a-c connects to a port connector 115a-c that is adapted for providing liquid intravenously. Preferably, the port connectors 115a-c are vacuum ports so that when the port is opened, the inner lumen of the port is not exposed to the environment.

In some embodiments, the primary catheter 120 connects directly to a single port connector 115b. In this embodiment, there is no secondary catheters 110, and it is also not necessary to include manifold 130. In this embodiment, the primary cathether is connectable to a bag for providing liquids 300, which is described below.

In some instances, an antibiotic disc 260 is seated at the base of the push button mechanism 200. On the back side of the flexible, polymer sheath is an adhesive layer 150, which is covered with a removable backing, such as the type of removable backing that can be peeled away. Typically, the adhesive layer 150 and removable backing do not cover the antibiotic disc 260. The adhesive layer 150 is preferably a medical grade adhesive, as are known in the art. In some embodiments, a portion of the removable backing covering the hollow needle 210 can be removed separately, thereby allowing the apparatus to be adhered to the skin over the implantable port. Then, the remainder of the removable backing can be removed to expose the remainder of the adhesive, and the device is then adhered to the skin.

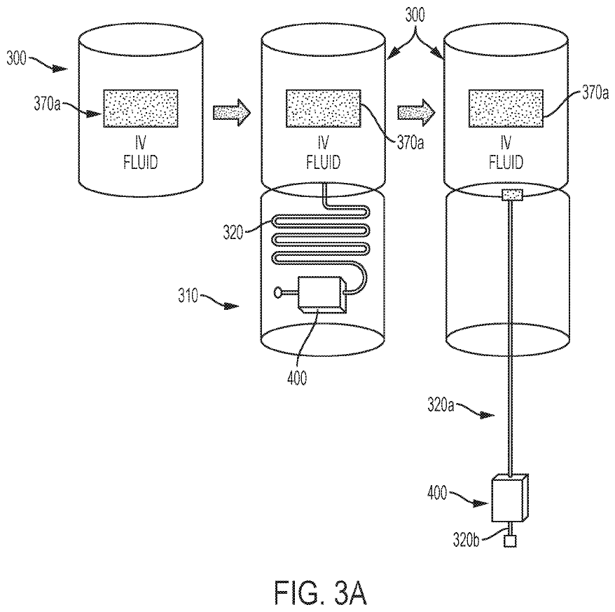

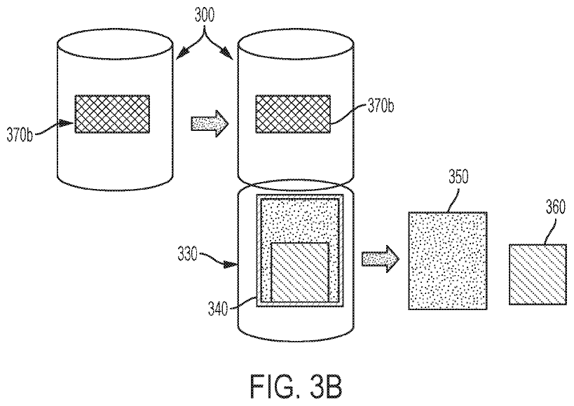

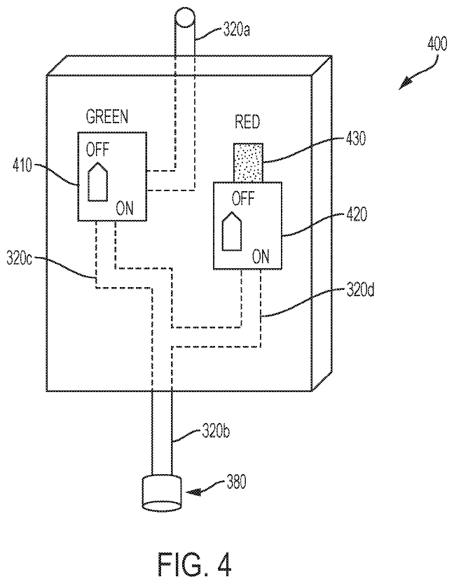

In order to provide liquid to a human, an apparatus holding liquids to be administered intravenously can be connected to the port connector 115a-c. An example of an apparatus holding the liquid is illustrated in FIGS. 3A and 3B. A liquid bag 300 has a first, front flap 310 that can be peeled down or pulled open to provide access to intravenous tubing 320a that connects the liquid bag 300 into a flush-flow box 400. By peeling down the front flap 310 and extending the intravenous tubing 320a-b, the liquid bag 300 is elevated so that gravitational forces cause fluid to flow through the intravenous tubing 320a towards the flush-flow box 400. The back side of the liquid bag 300 has a second, back flap 330 that can be peeled down or pulled open in a manner similar to the front flap 310. Peeling down the back flap 330 can reveal a compartment 340, which can be used to hold gloves 350 and an antiseptic wipe 360. The front and back sides of the liquid bag 300 can have content labels 370a-b, upon which pertinent labeling information can be provided.

The flush-flow box 400 can be used to flush the intravenous tubing 320 and to aspirate the implanted port. An intravenous tubing 320a enters the flush-flow box 400 and connects to a first switch 410. An intravenous tubing 320c also connects to the first switch 410. Typically, the intravenous tubing 320a connects to the first switch 410 above the intravenous tubing 320c. The intravenous tubing 320c is in fluid communication with intravenous tubing section 320b and 320d, which connects into second switch 420. In some embodiments, the first and second switches (410, 420) are referred to as stopcocks. A connector cap 380 is located at the end of the intravenous tubing 320b and connects the intravenous tubing 320b to one of the port connectors 115a-c.

The flush-flow box is first primed with fluid and then aspirated as follows. First, connector cap 380 is removed from intravenous tubing 320b. The first switch 410 is initially in an OFF position. Turning the first switch 410 to an ON position permits liquid to flow down through intravenous tube 320a and into the flush-flow box 400. Once the intravenous tubing 320a,c,b is filled with liquid, the first switch 410 is turned to an OFF position. Then, the intravenous tubing 320b is connected to one of the port connectors 115a-c. The second switch 420 has a vacuum capability and is initially in an OFF position. Turning the second switch to an ON position aspirates the implanted port by allowing blood, liquid, and air in intravenous tubing 320d and 320b to move upward through the second switch 420 and fill reservoir 430. Once reservoir 430 has filled and turned red, the second switch 420 is turned to an OFF position. Then, the first switch 410 can be turned to the ON position, thereby permitting liquid to flow through the intravenous tubing 320a,c,b and into one of the port connectors 115a-c, thereby establishing liquid communication from the liquid bag 300 to the implanted port. So long as the liquid bag 300 remains elevated relative to the implanted port, liquid will continue to flow from the from the liquid bag 300 into the human.

In order to use the device, an implanted port is first surgically installed into a venous blood vessel, usually a central venous blood vessel, of a human. The skin on the abdominal wall directly above the implanted port can be cleaned with an antiseptic wipe 360. The apparatus 100 for administering fluids is then attached to the body by removing the removable backing to expose the adhesive layer 150 and then adhering the apparatus 100 to the body so that the push-button mechanism 200 is centered above the implanted port.

When rapid intravenous administration of liquids is desired, the first, front flap 310 of the liquid bag 300 is peeled down or pulled open to provide access to intravenous tubing 320a-b. The device is then primed and aspirated using the flush-flow box, as described above. The cover 220 is removed, and the push button 205 is depressed, which causes hollow needle 210 to be advanced through the skin over the implanted port. If the push button has a channel connecting the hollow needle 210 to a reservoir, blood flows up to the reservoir via a capillary mechanism. The presence of blood in the reservoir 230 indicates that fluid communication has been established between the hollow needle 210 and the reservoir, and that the hollow needle 210 has punctured the implanted port. Then switch 410 is turned to the ON position, thereby permitting liquid to flow from the liquid bag 300 into the human via the implanted port.

The liquids administered from multiple liquid bags 300 can be the same or different. In some instances, multiple liquid bags 300 can be connected to the port connectors 115a-c, thereby allowing different liquids to be administered. For example, one liquid bag can have saline, another can have blood, and a third can have medicine. Examples of liquid that can be administered include saline, blood plasma or serum, blood substitutes, and antibiotics or other medicines.

### EQUIVALENTS

While this invention has been particularly shown and described with references to example embodiments thereof, it will be understood by those skilled in the art that various changes in form and details may be made therein without departing from the scope of the invention encompassed by the appended claims.

1. An apparatus for administering liquids to a human intravenously via an implanted port, the apparatus comprising:
a) a primary catheter in fluid communication with a hollow needle and connectable to a bag for providing liquid;
b) a push-button mechanism coupled to the hollow needle and configured so that depressing the push button mechanism inserts the hollow needle into the implanted port to provide liquid communication from the primary catheter, via the hollow needle, to the implanted port; and
c) the primary catheter embedded within a flexible, polymer sheath, wherein the flexible polymer sheath has a removable cover on a front surface to protect the push button mechanism and adhesive on at least a portion of the back surface, the adhesive being covered with a removable backing.

2. The apparatus of claim 1, further comprising multiple secondary catheters in fluid communication with the primary catheter via a manifold.

3. The apparatus of claim 2, wherein an inner surface of one or more of the primary and secondary catheters is impregnated with a heparin solution.

4. The apparatus of claim 3, wherein there are two or three secondary catheters.

5. The apparatus of claim 2, wherein one or more of the primary and secondary catheters is formed of silicone or polyurethane.

6. The apparatus of claim 4, wherein each lumen of the multiple catheter lumens has a port connector.

7. The apparatus of claim 6, wherein the port connector is a vacuum port connector.

8. The apparatus of claim 1, wherein the push button mechanism couples the hollow needle to a push button by a spring, wherein the spring is coupled to a locking component and configured to lock the needle into the implanted port when the push button is depressed.

9. The apparatus of claim 8, wherein the push button mechanism further comprises an antibiotic disc seated at the base of the push button mechanism and within the push button mechanism.

10. The apparatus of claim 9, wherein the adhesive does not cover the antibiotic disc.

11. The apparatus of claim 1, wherein the hollow needle has a small hole on an upper surface that is connected to a reservoir near the top of the push button via a channel so that blood can flow from the hollow needle to the reservoir.

12. The apparatus of claim 1, wherein the removable backing has at least first and second sections that can be separately removed, wherein the first section covers a portion of the adhesive configured to adhere the antibiotic disc to the skin directly over the implanted port, and wherein the second section covers a portion of the adhesive configured to adhere to skin surrounding the implanted port.

13. An apparatus for providing liquid intravenously to a human, the apparatus comprising:
a) a liquid bag having a first, front flap that is configured to be pulled open to provide access to intravenous tubing that is connected from the liquid bag into a flush-flow box, wherein the flush-flow box has an entry pathway in liquid communication with a first switch, and a second pathway in liquid communication with a second switch, and a common outlet tubing in liquid communication with both switches; and
b) a second, back flap that is configured to be opened to provide access to a rear compartment.

14. The apparatus of claim 13, wherein the intravenous tubing connected from the bag to the flush-flow box connected to the first switch operates to flush the intravenous tubing and provide liquid to flow to a port.

15. The apparatus of claim 14, wherein the second switch in the flush-flow box has a vacuum component, connects to the common outlet tubing, and operates to aspirate the port prior to infusion to verify proper venous access.

16. The apparatus of claim 13, wherein the rear compartment further includes one or more of gloves and an antiseptic wipe.

17. The apparatus of claim 1, wherein
a screw-top cap covers a button of the push-button mechanism.

18. The push-button mechanism of claim 17, wherein the housing is cylindrical.

19-21. (canceled)

22. A device comprising:
a) an apparatus for administering liquids to a human intravenously via an implanted port, the apparatus comprising:
i) a primary catheter in fluid communication with multiple secondary catheters via a manifold, the primary catheter in fluid communication with a hollow needle and connectable to a bag for providing liquid;
ii) a push-button mechanism coupled to the hollow needle and configured so that depressing the push button mechanism inserts the hollow needle into the implanted port to provide liquid communication from the primary catheter, via the hollow needle, to the implanted port; and
iii) the primary catheter, secondary catheter, and manifold embedded within a flexible, polymer sheath, wherein the flexible polymer sheath has a removable cover on a front surface to protect the push button mechanism and adhesive on at least a portion of the back surface, the adhesive being covered with a removable backing;
b) a liquid bag comprising:
i) a first, front flap that is configured to be pulled open to provide access to intravenous tubing that is connected from the liquid bag into a flush-flow box, wherein the flush-flow box has an entry pathway in liquid communication with a first switch, and a second pathway in liquid communication with a second switch, and a common outlet tubing in liquid communication with both switches; and
ii) a second, back flap that is configured to be opened to provide access to a rear compartment.
